(19) United States
(12) Patent Application Publication
(10) Pub. No.: US 2007/0088264 A1
(43) Pub. Date: Apr. 19, 2007

(54) DEVICE FOR INTRODUCING AN ACTIVE INGREDIENT INTO THE SKIN

(76) Inventor: Horst LIEBL, Zelsheim/Alsace (FR)

Correspondence Address: ROBERTS, MLOTKOWSKI & HOBBES, P. O. BOX 10064, MCLEAN, VA, 22102-8064, US

(21) Appl. No.: 11/470,524

(22) Filed: Sep. 6, 2006

Related U.S. Application Data
(60) Provisional application No. 60/716,493, filed on Sep. 14, 2005.

(51) Int. Cl. A61M 5/00

(52) U.S. Cl. 604/117

(57) ABSTRACT

A device for introducing an active ingredient into the skin. The device has at least two rollers mounted so that they are rotatable around their longitudinal axis, from whose outer peripheral surface multiple needles project radially outward. In order to make application of the device over a large area easier, the rollers are situated parallel to one another and laterally offset relative are situated parallel to one another with axially neighboring rollers being laterally offset relative to one another such that a projection of one of the rollers perpendicular to the longitudinal axis thereof comes to rest on an extension of the longitudinal axis of the neighboring parallelly situated roller without needle-bearing areas of the outer peripheral surfaces of both rollers overlapping in an axial direction.

### BACKGROUND OF THE INVENTION

1. Field of the Invention

The present invention relates to a device for introducing an active ingredient into the skin having rollers mounted so they are rotatable around their longitudinal axis, and on whose outer peripheral surface there are multiple needles which, at least in a tip area, project from the outer peripheral surface, taper outward to a point, project radially outward.

2. Description of Related Art

In the structure of the skin, one basically distinguishes between the epidermis and the lower-lying dermis. The epidermis, which is approximately 0.03 to 0.1 mm thick, is typically divided into five further layers of skin and is predominantly composed of dead epithelial cells. New epithelial cells are continuously formed by the body, so that the epidermis is constantly renewed. The basic desirable natural barrier function of the epidermis, such as shielding the lower layers of skin from external bacterial influence, physical toxins, etc., is opposed by the disadvantage of strongly reduced penetrating ability of topically applied active ingredients.

In particular, in dermatology and cosmetics, surface application of active ingredients, typically in the form of dermatics, such as salves and creams, containing active ingredients is very widespread for introducing active ingredient into the skin. The active ingredients contained therein may counteract skin aging, for example.

Aging of the skin is expressed, inter alia, by a loss of transparency, moisture, and tautness. The symptoms may be attributed to the natural reduction of the thickness of the layers of skin, the degradation of fatty tissue in the deeper layers of skin, but also to shrinkage and hardening of collagen fibers with increasing age.

It is known that multiple active ingredients excite and encourage the revitalizing processes, such as the formation of new collagen tissue, in the lower-lying dermis. In order that an active ingredient applied to the skin may reach the location of action, however, or into the dermis, the epidermis, which prevents the penetration of substances useful for the skin in its natural function, must be overcome.

Clinical studies have shown that more than 99.7% of active ingredients applied in a typical way to the skin normally remain in the epidermis. Correspondingly, only at most 0.3% of the active substances penetrate the epidermis and reach the dermis, where they may unfold their desired effect.

In order to increase the active ingredient concentration in the dermis, the proportion of active ingredient in the salves, creams, etc. used is frequently increased. However, this method typically causes a normally undesired, dramatically elevated active ingredient accumulation in the epidermis, because of which side effects, which may extend up to destruction of the epidermal layers, may occur. A loss of the outer protective armor of the skin has grave consequences, however. Thus, a skin surface affected in this way nearly always displays oversensitivity in relation to external influences such as UV radiation, dryness, etc.

Alternatively, additives such as liposomes, acids, etc. are added to topical formulations. In addition to the production of corresponding topical agents, which is frequently technologically very complex, these additives normally also result in destruction of the epidermal layer, so that the above-mentioned disadvantages are also to be observed here.

A further possibility is the mechanical removal of the epidermis in order to make access to the dermis easier for the active ingredients. For this purpose, the upper layers of skin are rasped away using diamond burrs, vaporized using lasers, etc. These drastic alternatives are typically extremely unpleasant and painful for the person treated, so that an accompanying analgesic treatment and wound care, which is frequently very tedious, are necessary here. In addition, this procedure also results in a temporary loss of the natural protective function of the skin. Therefore, a skin surface treated in this way is also extraordinarily sensitive to external factors.

With the needle roller described in German Patent Application DE 100 63 634 A1 and corresponding International Application Publication WO 02/49711 A1, the present applicant has already developed a method by which active ingredient penetration into the dermis may be increased by more than fortyfold in comparison to untreated skin, without disadvantageously damaging the skin surface. In addition, the treatment is practically painless for the affected person. In rare cases, slight skin irritations may be observed, but these disappear completely again after only a few days. The natural protective function of the epidermis, in contrast, is completely available again nearly immediately after the treatment, since puncture channels caused on the skin by the needles of the needle roller typically close again after a few minutes. Furthermore, the needle roller allows a defined and reproducible penetration depth of the needles into the skin to be ensured, which means a significant increase in operating comfort and safety for both the treating person and also the treated person.

To treat the skin surface, the rotatably mounted roller of the needle roller is moved manually over a skin region on which an active ingredient or a mixture containing active ingredient either has been applied or will be applied. In order to guide the needle roller described in German Patent Application DE 100 63 634 A1 over the skin, the roller is attached by a fork to a handle. Multiple needles project radially outward from the outer peripheral surface of the roller, which needles penetrate into the skin, and thus, open tiny channels through which the applied active ingredient may pass the epidermis and finally penetrate into deeper layers of skin. The channels remain passable to an active ingredient long enough that further active ingredients may penetrate successively into the skin. Supporting measures, such as massaging the skin or similar measures, may further encourage the active ingredient penetration. In addition, the needle roller has the special advantage that, after the needles have already penetrated completely into the skin using the length projecting from the outer peripheral surface of the roller, the roller outer surface of the roller rests on the skin. The needles may thus only penetrate exclusively up to a predefined maximum depth into the skin surface, independently of the level of pressure exerted on the roller.

In order to make penetration into the skin easier, the needles taper outward to a point, at least in a tip area projecting beyond the outer peripheral surface. Furthermore, the needles are distributed regularly and uniformly on the roller, through which a targeted and reproducible control of the number of puncture channels is made possible via the frequency of the movements of the needle roller over the skin. Direct influence on the active ingredient penetration may thus also be obtained through variation of the number of punctures per unit area on the skin.

According to clinical tests, the needle roller is particularly suitable for treating cellulite (“orange peel skin”), which often occurs over a large area, in connection with specific active ingredients and may achieve clear treatment success in a comparatively short time here.

However, the treatment of a large skin surface, such as an upper thigh, by the needle roller of German Patent Application DE 100 63 634 A1 is comparatively time-consuming because the roller has a width of only 1 to 2 cm, so that the resulting strip widths of the areas penetrated by the needles are correspondingly limited to a width of 1 to 2 cm. The needle roller of German Patent Application DE 100 63 634 A1 is thus less suitable for a large-area application.

### SUMMARY OF THE INVENTION

In view of the foregoing, it is a primary object of the present invention to provide a device which allows uniform introduction of an active ingredient into large-area skin regions. In addition, the device must be designed in such a way that safe and reliable treatment of the skin surface is ensured.

Proceeding from German Patent Application DE 100 63 634 A1, to achieve this object, the possibility exists of overcoming the disadvantages described by enlarging the axial roller length of the needle roller. This approach to achieving the object has the problem that the resulting very broad roller is subjected to significant pressures and/or bending torques during the application to the skin. Therefore, there is an increased risk that the roller will bend along its axis, because of which it may no longer be rolled uniformly along the skin. If the roller bends even more strongly, the danger exists that the roller will be pressed out of the fork. Furthermore, a permanent bend of the roller loosens the adhesive bonds between the individual roller disks of DE 100 63 634 A1, so that the needles lose their defined positioning and no longer penetrate into the skin surface in a predefined way. Due to the loosened bond between the roller disks, the probability is significantly increased that the roller will break apart during a treatment. In summary, a significant risk of injury exists in a roller broadened in this way due to needles which remain stuck in the skin, penetrate deeper than desired into the skin, and cause undefined cuts and cracks on the skin, for example. Furthermore, the rollers suddenly slipping out of the fork or even breaking may trigger further significant injuries.

In order to effectively lower the described increased injury potential from using a corresponding axially lengthened roller, the constructive requirements increase in a disproportionately great manner. Thus, inter alia, a comparatively complex and costly precisely fitted axle is required to be able to additionally laterally stabilize the roller disks. Furthermore, the fork must be designed as significantly more massive in order to be able to absorb the resulting forces without deformation, allow exact guiding of the roller, and prevent the roller from jumping out of the fork unintentionally. The special requirements described for the technical implementation of such a wide needle roller would result in a drastic increase of the production costs overall.

Rather, the object is achieved by the device in which at least two said rollers are situated parallel to one another with axially neighboring rollers being laterally offset relative to one another such that a projection of one of the rollers perpendicular to the longitudinal axis thereof comes to rest on an extension of the longitudinal axis of the neighboring parallelly situated roller without needle-bearing areas of the outer peripheral surfaces of both rollers overlapping in an axial direction.

In a first aspect, the present invention relates to a device for introducing an active ingredient into the skin which comprises at least two rollers provided with needles, the basic construction of the needles and the rollers being that disclosed in German Patent Application DE 100 63 634 A1. The rollers of a device according to the present invention are situated parallel to one another in order to allow continuous rolling on the skin surface. As the rollers are rolled along the skin, the needles projecting beyond the outer peripheral surfaces of the rollers penetrate into the epidermis and leave behind strip-shaped areas, which are typically not visible, having tiny punctures on the skin surface. The strip-shaped areas are referred to in the following as roller strips. Each of the at least two rollers leaves at least one roller strip behind on the skin, whose width corresponds to the length of the axial outer edge of the corresponding roller. The rollers are not only situated parallel to one another, but are also laterally offset relative to one another in such a way that upon a projection of one of the rollers perpendicular to its longitudinal axis comes to rest on an extension of the longitudinal axis of the axially neighboring parallel roller(s) without the areas of the outer peripheral surfaces of both rollers from which the needles project outward overlapping in an axial direction. This system has the advantage that, through the lateral offset of the rollers in the rolling direction, the individual roller strips do not overlap as the device is rolled along the skin, but rather run parallel to one another. The maximum overall roller width of the device may thus be achieved using the available rollers. The basic construction of the rollers is comparable to the rollers already proven from German Patent Application DE 100 63 634 A1.

The rollers are preferably situated laterally offset relative to one another in such a way that the individual roller strips combine to form an essentially coherent area. This special arrangement corresponds to the effect of a single very wide roller, but the constructive disadvantages described above which are associated with a strongly widened roller do not occur. In contrast, in comparison to the needle roller described in German Patent Application DE 100 63 634 A1, the time required to uniformly roll over a defined skin region using the device and provide it with puncture channels is significantly reduced.

If the device has more than two rollers, they are preferably situated along only two longitudinal axes in order to achieve a compact construction of the device. More than two rollers are very especially preferably situated in such a way that the longitudinal axes of odd numbered rollers, i.e., 1^{st}, 3^{rd}, etc. (and even numbered rollers, i.e., 2^{nd}, 4^{th}, etc., if more that three rollers are used) are coaxially arranged. Therefore, every roller attached to the device which exceeds two rollers is situated coaxially to a roller beyond the next roller in the axial direction. An especially compact embodiment of the device may thus be achieved because the rollers may be situated in a very space-saving way and, nonetheless, the roller strips lie very close to one another or combine to form a nearly continuous overall surface.

An embodiment having a total of three rollers has been shown to be especially advantageous, the rollers being situated in a V-shaped arrangement relative to one another. The longitudinal axis of a laterally offset roller runs parallel to the longitudinal axis of the two other rollers, which are situated coaxially relative to one another. Furthermore, the one offset parallel roller is located between the two other rollers. Since the width of a single roller is preferably in a range from 1 to 3 cm and particularly 2 cm, the overall roller strip in the preferred continuous arrangement of three rollers has a total width of 3 to 9 cm and particularly 6 cm. It has been shown that using these preferred dimensions of the rollers, devices are obtained which are distinguished in their compactness by especially good handling, since they are comfortable to hold in one hand. In addition, the required treatment time is still reduced by a factor of three in comparison to the needle roller of German Patent Application DE 100 63 634 A1. Moreover, the device does not tilt due to the V-shaped arrangement of the rollers, but rather stands stably on the skin surface. This property allows guiding of the device on the skin practically without wobbling, in addition, the device may also be set aside without it tilting over and the danger of unintended engagement in the needle rollers arising.

In addition, it is advantageous to implement all rollers of the device having identical constructions, through which the individual rollers may be produced in the same way and the production costs of the device may be significantly lowered.

In order to ensure homogeneous penetration improvement over the entire skin region to be treated, it is recommended that the needles be distributed uniformly over the outer peripheral surface of each individual roller, but also uniformly over the entirety of the rollers. For this purpose, the rollers of the device, as is already known from German Patent Application DE 100 63 634 A1, are expediently each constructed from multiple parallel disks, between which the needles are embedded at regular intervals. The disks advantageously have depressions for this purpose, which extend outward in the radial direction on at least one of the sides of the disks. In addition, it is advantageous if the length of the needles which projects beyond the outer peripheral surfaces of the rollers lies in defined limits, so that all needles penetrate equally deep into the skin surface. In order to prevent slipping of the needles into the interior of the roller, a stop is expediently provided on the inner end of the depressions for each of the needles for this purpose.

The needles preferably project at equal length and particularly at a length of 0.12 to 1.5 mm beyond the outer peripheral surfaces of the rollers. In order to make penetration of the needles into the skin easier, these needles are expediently tapered outward to a point in the tip area that projects from the outer peripheral surface of the rollers. It is advantageous if round, atraumatic needles are used, which push the epithelial cells of the epidermis apart upon penetration of the needles into the skin. In contrast, traumatically acting trocars, which are widespread in medicine and which slice the tissue, because of which the epidermis requires a significantly longer regeneration phase in order to recover its protective function in its entirety, are not preferred. The needle tips are expediently as thin as possible without the danger of breaking of the needles arising. The optimum diameter of the needle tips which may be implemented is largely a function of the material used. Furthermore, the diameter of the needle tips may be optimized to the type of the application. Needle tips having a diameter in the tip range from 0.05 mm to 0.1 mm have been shown to be advantageous and 0.08 mm has been shown to be very especially advantageous. Outside the tip area, the needles expediently have a diameter of 0.15 to 0.3 mm. The needles of the device according to the present invention are preferably made of metal and particularly stainless steel. The tips of the needles are typically ground and subsequently electroplated.

In a roller constructed from multiple disks, the needles are preferably first glued in the depressions provided on the disks in order to additionally stabilize the needles in their position. Subsequently, the disks are bonded parallel to one another. An adhesive may also be used for this purpose. In order that the disks have the desired orientation relative to one another, it is especially preferable, in accordance with the present invention, to provide at least one centering projection on at least one of the surfaces of the disk. This at least one centering projection corresponds to a corresponding centering depression on the surface of the neighboring disk. It is especially expedient if the centering projection is used simultaneously as a rear stop for the needles. Such centering projections which result simultaneously in centering of neighboring disks and setting of a predefined angular offset of the needles of the neighboring disks to one another are especially preferred. For example, a projection in the form of a toothed collar which projects in the center of the disk is suitable for this purpose. A corresponding depression in the form of a toothed collar is implemented in a neighboring disk and receives the projection of the other disk in a formfitting way.

Neighboring disks of a roller may be situated on one another so that the needles of all neighboring disks lie on lines which run parallel to the roller longitudinal axis. Alternatively, it is possible to situate needles of neighboring disks on gaps. The lateral offset of the rollers relative to one another is advantageously selected so that the positioning pattern of the needles on one roller continues nearly without a transition on the neighboring roller in the axial direction.

The number of needles on a roller of predefined size may be controlled via the number of disks—i.e., via the disk thickness—and the number of needles per disk. It has been shown that too dense a population of the rollers with needles may have a negative effect, since a type of “bed of nails effect” occurs and the needles may only penetrate into the skin with difficulty. Suitable population numbers are achieved, for example, if the needles per disk are situated on straight lines which form an angle of 15 or 30° out from the center point of the disk. This corresponds to 24 or 12 needles per disk. Suitable disk thicknesses are, for example, 2 to 3 mm, particularly 2.5 mm.

The number of disks used is expediently oriented to the intended use of the device according to the present invention. Depending on the size of the area of skin to be treated, for example, 2 to 3 disks per roller may suffice, but 10 or more disks may also be used. Limits only exist here in regard to the absolutely required maintenance of the bending strength or the stability of the individual rollers obtained. Typically, the individual rollers are constructed from 7 disks. End disks may be used as front termini of the disk system, which result in a smooth axial terminus of the roller thus resulting, for example.

The diameter of a roller or the disks forming it may also vary over a broad range. By varying the size of the diameter, influence may also be exerted on the spacing of neighboring needles. Moreover, it is possible to set the length of the projection of the needles beyond the outer circumference by varying the size of the diameter, and thus, control the penetration depth of the needles. In this way, without changing the needle length, devices having different penetration depths of the needles may be produced. Vice versa, it is possible to keep the diameter of the roller or the disks forming the roller constant and instead vary the needle length and obtain devices having different penetration depths of the needles in this way. For example, roller diameters of 1 to 5 cm may be used with rollers having a diameter of approximately 2 to 3 cm being especially suitable.

In order that the at least two rollers of a device according to the present invention may be guided comfortably and safely over the skin surface, the device preferably has a housing which may be constructed from one or more parts. The housing shields the rollers on the side facing away from the skin, so that the operator is protected from unintentionally touching the needle tips with the hand or palm. In addition, the hygienic safety of the operator is increased. This is particularly advantageous if the treated person is not identical to the treating person. Preferably, the housing is constructed in such a way that the operator rests the interior of his hand on the housing top and/or may additionally grip around the housing laterally with his thumb and an additional finger. This preferred embodiment may be held very securely and thus allows controlled guiding of the rollers over the skin surface. It is very especially preferable to make the basic housing shape similar to the shape of a computer mouse. Such an anatomically shaped housing represents a significant convenience for the personnel who must operate the device daily and/or over longer periods of time in particular. In addition, this form is noticeably handy and compact. The top of such a housing is typically curved slightly upward in the middle area and drops off flatter in the area facing toward the front side than in the direction of the diametrically opposite rear edge and the two lateral areas.

It is expedient to connect the rollers to the housing in such a way that at least the tip area of the needles projects beyond a plane spanned by the lower edge of the housing, so that the needles may penetrate into the skin surface. However, in order to allow rolling of the device on the skin without problems, the lower edges of the outer peripheral surfaces of the rollers preferably project at a height of 2 mm to 8 mm, and particularly, 5 mm beyond the lower edge of the housing.

All typical systems which allow free rotatability of the rollers around their longitudinal axes may, in principle, be used for the rotatable mounting of the rollers. Thus, axial extensions may also be integrated into the end disks, which are received by bearings mounted on the housing, for example. Alternatively, the rollers may also have lateral indentations, into which suitable protrusions attached to the housing project. In this regard, the use of axles which run through the rollers along their longitudinal axes is advantageous. Such axles provide the rollers with additional stability and/or increased bending strength and also relieve the connections of the individual roller disks to one another. It is possible for multiple rollers to be attached to the same axle. However, each roller preferably has a separate axle in order to reduce the technical requirements on the axles and to keep the loading forces on each individual axle as low as possible. In order to be able to lead an axle through the rollers, the individual roller disks have a through hole in their center point, whose diameter expediently essentially corresponds to the diameter of the axles. In principle, the axle diameter is variable, but it is preferably between 1 and 3 mm and particularly 2 mm. In general, all materials which fulfill the practical requirements in regard to stability and bending strength are suitable for producing the axles. Thus, for example, it is possible to use plastic axles or axles made of a suitable metal. Stainless steel axles have been shown to be especially suitable for this purpose.

In order to connect the axles to the housing, it is especially advantageous if the axles project on both axial sides of the roller. The resulting axle projections may now be received by typical bearings which are attached for this purpose to the housing. Thus, for example, it is possible for notches or cutouts to be provided on the housing edge, in which the axle projections engage. Alternatively, the axles may be guided through corresponding holes on the housing. Furthermore, it is possible to mount the axles between housing parts lying one on top of another. Additional grooves and notches may be provided for this purpose on the housing parts. In principle, the axles may be connected rotatably or fixed to the housing. If the axles are mounted rotatably on the housing, the rollers are expediently connected fixed to the axles. In contrast, if the axles are attached fixed to the housing, the rollers each rotate around the axle running through the relevant roller.

However, struts, which are situated in pairs, project in the direction of the side facing toward the skin, and are spaced apart from one another have been shown to be especially suitable for attaching the rollers to the housing, since these reliably limit axial play of the roller and optimally dissipate the resulting forces into the housing. A strut pair is typically spaced apart from one another by one roller width. Each strut pair has special recesses for receiving the axle projections, into which the axle sections of the rollers projecting axially out of a roller may be inserted. The recess is preferably attached on the front face of the strut facing toward the skin, because of which the assembly of the device is made easier. In order to secure the axle horizontally in its position, the recess advantageously has a width which essentially corresponds to the diameter of the axle projection. The dimensions of the struts are designed in such a way that they may absorb the pressure exerted during rolling of the device on the skin. The struts preferably have a thickness of 1 mm to 4 mm and particularly 2 mm in the axial direction of the rollers. The struts have a preferred width of 5 mm to 20 mm and especially preferably 10 mm perpendicular to the axial direction. The struts are advantageously attached in the interior of the device to the top of the housing and run vertically in the direction of the skin surface. This allows especially stable positioning, since the struts may optimally absorb and relay the resulting pressure.

In a special embodiment of the device, the diameter of the axle projections is less than the diameter of the remaining axles. In this case, the axle diameter of the axle projection is preferably in a range between 0.5 mm and 2 mm and particularly 1 mm. The struts, whose recesses have a width which essentially corresponds to the axle diameter of the axle projection, prevent the axles from slipping laterally out of the recesses in this embodiment. The axle diameter of the middle part of the axle is too wide to fit into the recess of the strut.

Moreover, it is advisable to attach the axles in the recesses of the struts in such a way that the axle projections do not fall out of the recesses of the struts during transport or use of the device. For this purpose, the recesses of the struts may be designed, for example, so that the axle projections engage in the recesses. However, in this special procedure, the danger exists that the axle projections will bend as they are pressed in until they engage. In addition, pointed needles project beyond the entire outer surface of the roller, because of which controlled contact pressure is made more difficult.

Therefore, it has been shown to be advantageous to secure the axle projections in the recesses through fixing means. These are pushed into the remaining free space of the recess onto the axle projection after the axle projection has been introduced into the recess. In order that the fixing means are later permanently connected to the strut, they are preferably glued to the strut. Furthermore, if the recesses lie on the side of the strut facing away from the skin, the fixing means are inserted from the direction of the skin surface into the recesses.

As the device according to the present invention is rolled along the skin surface, the axle projections thus do not exert any pressure on the fixing means. The material strain of the attachment of the fixing means is therefore extremely low with this preferred positioning of the recesses.

The shape of the individual fixing means may vary. Thus, the two diametrically opposite sides of the fixing means which are in contact with the side walls of the recesses may run trapezoidally. The wedge thus obtained is subsequently easy to insert into the recess. Typically, however, a cuboid shape having a width which essentially corresponds to the width of the recess is preferred, since, independently of how far the fixing means project into the recess, it may be pushed up to shortly before the axle.

The individual fixing means are typically small and unhandy. Therefore, it is suggested that multiple or all of the required fixing means be integrated in a common cohesive component such as a strut, frame, or a housing lower part. All required fixing means of a device according to the present invention may thus be produced in a single step with the corresponding housing part and inserted therewith into the recesses of the struts, which are preferably attached to the corresponding housing upper part for this purpose. Through the connection of the fixing means to the lower part without a transition, the connection of the fixing means to the recesses additionally achieves significantly higher stability.

In order to make it easier to roll the device uniformly along the skin surface, it is possible to close the housing on the bottom with a preferably smooth surface as much as possible. For this purpose, the surface has individual openings through which the rollers may penetrate. This refinement is particularly advantageous if the rollers project only a very small distance beyond the bottom of the housing and therefore a part of the bottom edge of the housing presses very quickly against the skin surface when the device is pressed against the skin, through which the movement flow is obstructed. It is advisable to design the opening as small as possible and as large as necessary. Thus, unintentional pinching of a skin fold between the edge of the opening and a roller, which would doubtlessly be very unpleasant for the treated person, may be nearly precluded. A closure of the housing bottom is especially preferably produced by a separate lower part. For this purpose, the lower part, which already has a smooth outer surface and the corresponding openings for the rollers, is attached to the bottom of the housing upper part.

If the housing comprises multiple parts and particularly an upper part and a lower part, it is advisable to connect the housing parts so solidly to one another that the housing of the device has a sufficiently high stability in use. In general, the typical connection methods are suitable for this purpose. Thus, the housing parts may be clamped, glued, or welded to one another. Furthermore, it is possible to screw, rivet, etc., the individual components of the housing to one another. Preferably, however, the housing parts are attached to one another using typical catch connections. For this purpose, one housing part has notches, for example, in which projections attached to the neighboring housing part engage and snap. This type of connection is preferable since the required notches and projections may already be integrated in the corresponding housing parts, so that no additional components are required. Moreover, such a connection may be produced very rapidly and easily by pressing the housing parts together. Although reversible connections between the housing parts are possible, the housing parts are preferably connected to one another in such a way that opening the housing part without destruction is not possible. This measure particularly avoids injuries due to individual parts lying around, such as the needle-equipped rollers.

Furthermore, the device according to the present invention may have holding and guide means in order to make the transport and use of the device safer. Thus, for example, a knob may be attached to the housing top. Moreover, it is possible to provide the housing surface with structures, such as nubs or grooves, in order to make it more difficult for it to slip off the guiding hand. In this context, however, a bow attached to the housing has been shown to be especially suitable. The device may be raised and transported without problems by the bow. Furthermore, the bow may effectively prevent unintentional falling down of the device during use. The bow is typically pivotable over the top of the housing so that the operating person may select whether or not the bow is erect. Arrangements in which the bow may be pivoted over the entire top of the housing are possible, as are arrangements which allow only partial pivotability of the bow. The bow is expediently attached to the housing in such a way that the pivot direction of the bow is oriented in the rolling direction of the rollers and the bow may thus stand transversely to the rolling direction.

In the erect state of the bow, referred to in the following as the operating position, the operator may grasp in the bow with his hand, so that the top of the device contacts the hand interior and the bow encloses the hand exterior of the operating hand. The bow is dimensioned correspondingly for this purpose. In the folded down position of the bow, referred to in the following as the storage position, the bow is moved transversely to the axial direction of the rollers. The bow is preferably attached to the housing, so that the device may also be held by one hand in the storage position of the bow. Furthermore, the device is now significantly smaller into its external dimensions, because of which space-saving storage is made possible.

The bow is attached to the device according to the present invention in a typical way. If the bow is not pivotable, bow and housing may be manufactured of a one-piece construction. Furthermore, such a bow may also be glued or welded on. It is also possible for the bow to have catch connections or eyes which may be pushed over the housing and subsequently produce a form fit with corresponding receiving elements attached to the housing. Preferably, however, the bow is connected pivotably to the device according to the present invention, in order to make the bow rapidly and easily available through erection and vice versa. For this purpose, for example, bolts, screws, etc., may produce a connection between bow and housing.

However, in order to reduce the number of individual parts required for the device according to the present invention, it is desirable to use a pivot mechanism which does not require any additional components for manufacturing the device. For this purpose, protrusions, typically in the form of half spheres, spherical caps, or cylinders are attached to the bow or housing, which are received by corresponding indentations or holes which are attached to the particular diametrically opposite component. This special type of attachment additionally allows the bow to be reversibly attached to the housing, and/or also to be removed again if desired by slightly bending the side parts of the bow apart without permanent damage.

Furthermore, a special transport container may be provided for storing a device according to the present invention. This has an interior which may entirely or at least partially accommodate the device. In an especially preferred embodiment, the transport container has supports which are attached to the floor and/or in the lower area of the side walls. When the device is introduced into the transport container, the lower edge of the housing of the device rests on the supports, the contact surface of the supports expediently being at least far enough from the floor surface of the transport container so that the needles projecting radially outward from the rollers do not contact the floor of the transport container. The needles are now completely enclosed by shielding parts and undesired contact of the needles by people, etc., is prevented. In addition, the needle tips are protected, since there is no direct contact of the needle tips with a hard substrate. In a further embodiment, the transport container is additionally used as a cleaning bath for the device. For this purpose, it is filled with appropriate cleaning and sterilization liquids, such as 3% hydrogen peroxide solution or 70% medical alcohol.

A fitted cover is additionally provided to cover the transport container. In addition to the function as a contamination protection during long storage of the device, this cover may particularly prevent the evaporation loss of the cleaning and sterilization liquids used.

In addition to the preferred use of stainless steel for producing the needles and the roller axles already cited, the remaining components of the device according to the present invention may each be manufactured from suitable materials. A plastic, particularly a plastic which may be injection molded, is preferably used for this purpose. In addition, however, it is also possible for individual parts to be made of metal and particularly stainless steel, for example. The plastics, metals, and cured adhesives used are preferably dermatologically harmless. Furthermore, the adhesives used are to be compatible with the corresponding plastics. For example, these may be cyanacrylate adhesives. Moreover, nearly arbitrary combinations of different suitable materials are conceivable. However, it is preferable for the materials used to be resistant to the conditions typically used for sterilization and cleaning. These include, for example, both sterilization methods using UV and gamma rays and also typical cleaning and sterilization liquids, such as aqueous hydrogen peroxide solutions and alcoholic dilutions.

The present invention is to be explained in greater detail in the following on the basis of the accompanying drawings.

### BRIEF DESCRIPTION OF THE DRAWINGS

FIG. 1a is a schematic side view in outline form of a preferred embodiment of the device according to the present invention,

FIG. 1b is a schematic frontal phantom view of the embodiment shown in FIG. 1a,

FIG. 1c is an illustration of the device according to FIG. 1a as seen from the skin contacting side,

FIG. 2a is a schematic illustration of an individual roller mounted on a strut pair,

FIG. 2b is a schematic illustration of a preferred implementation of the axles viewed perpendicular to the longitudinal axis of the axle,

FIG. 2c is a side view of a strut having fixing means,

FIG. 2d is a side view of a strut and a fixing means projecting from a bottom strut,

FIG. 3a is an front view of an exemplary disk surface of a disk equipped with needles,

FIG. 3b is a rear view of the disk shown in FIG. 3a,

FIG. 3c shows the surface of an end disk looking toward the interior of a roller,

FIG. 4a is a side view of a roller mounted in a strut having continuous rows of needles over all disks,

FIG. 4b shows a frontal view of the roller shown in FIG. 4a,

FIG. 5a is a top view of the bottom of a two-part housing,

FIG. 5b is a cross-section along line VII through a two-part housing,

FIG. 6a is a side phantom view of the device having bow in rest and transport positions,

FIG. 6b is a rear view of the device having bow in rest and transport positions,

FIG. 7 is a schematic illustration of the device in a transport container.

### DETAILED DESCRIPTION OF THE INVENTION

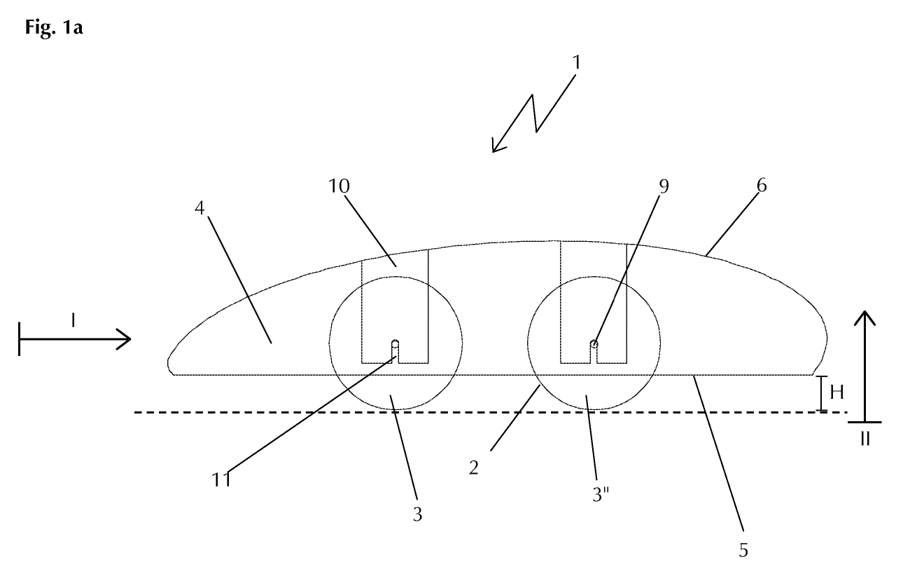

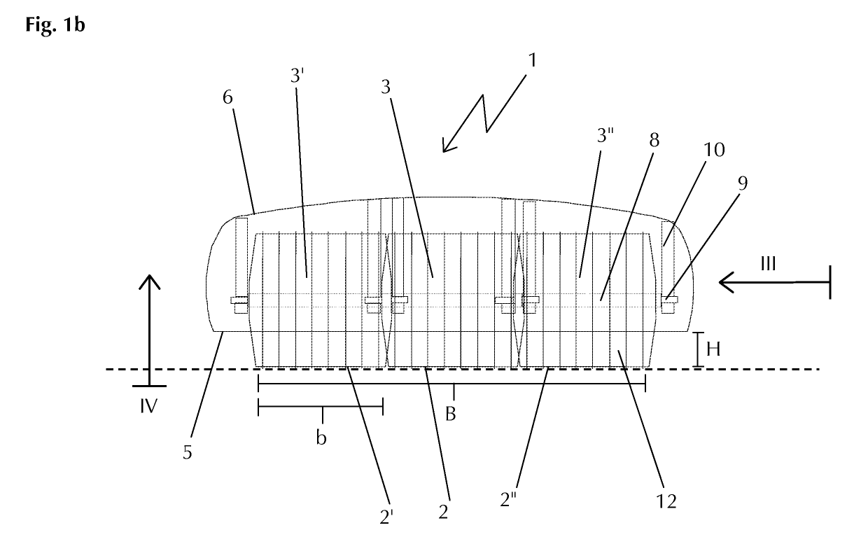

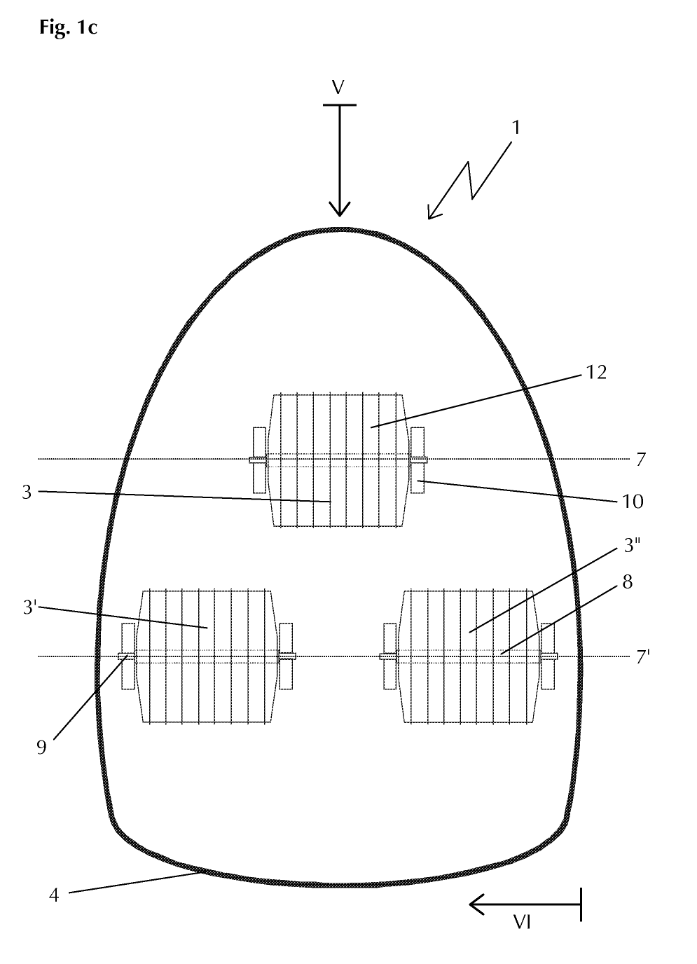

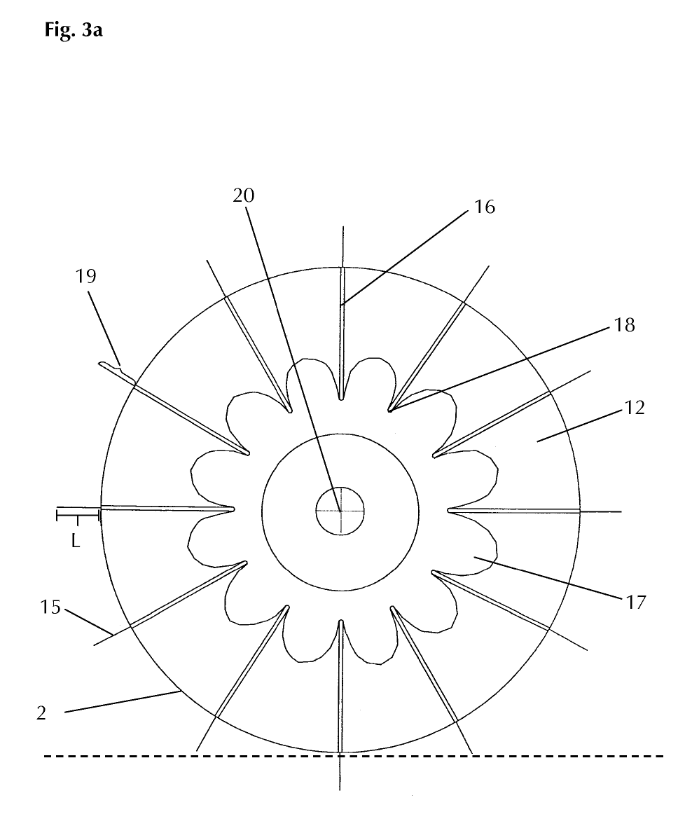

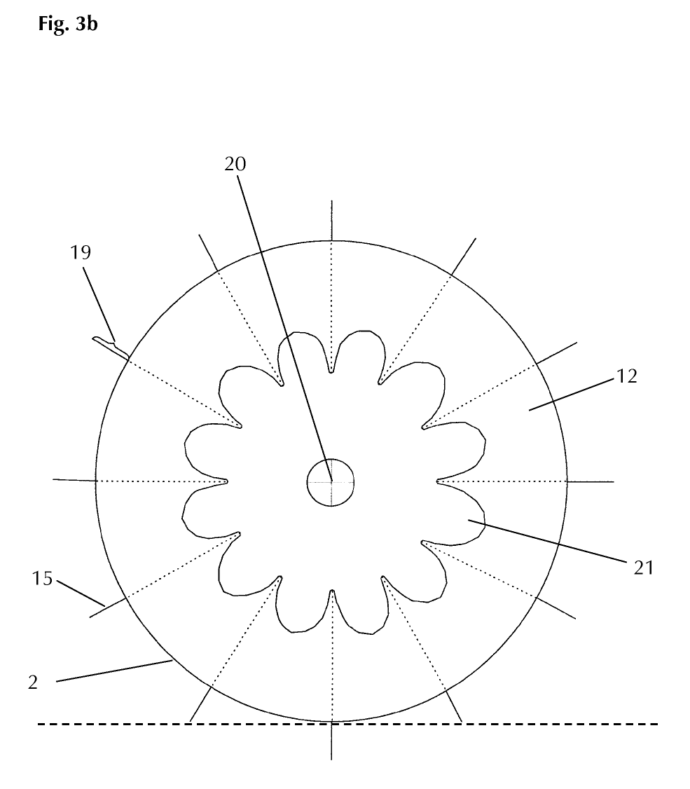

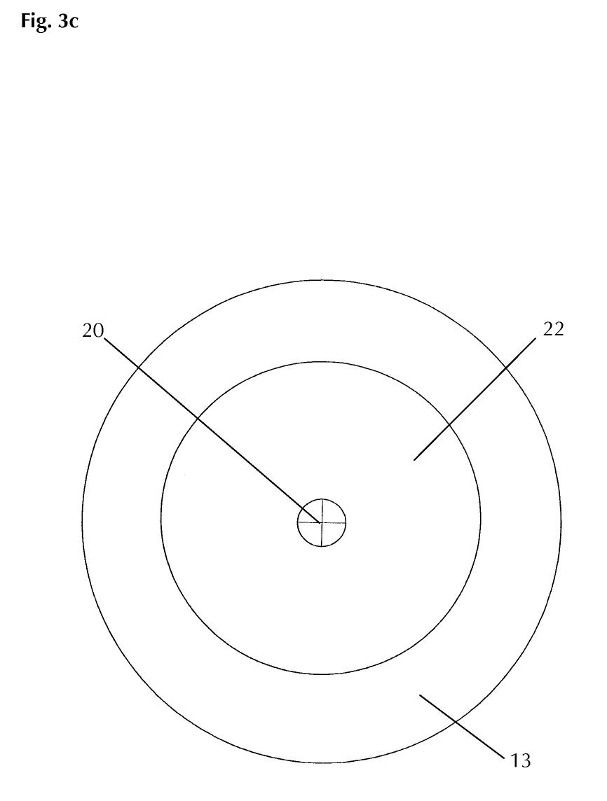

An especially preferred implementation of a device 1 according to the present invention is shown in FIGS. 1a, 1b, and 1c on the basis of views in which the arrows inscribed with Roman numerals indicate the relationships of FIG. 1a (III and VI), 1b (I and V), and 1c (II and IV) to one another. The needle tips projecting beyond the outer peripheral surfaces 2, 2′, and 2″ of the rollers 3, 3′, and 3″ are not shown in detail here for the sake of clarity. Rather, the more detailed construction of the rollers 3, 3′, and 3″ may be inferred from FIGS. 3a, 3b, and 3c.

The device 1 shown in FIGS. 1a, 1b, and 1c comprises a housing 4. The housing bottom 5 of the housing 4 faces toward the skin surface to be treated (shown as a dashed line in FIG. 1a and FIG. 1b), and the housing top 6 correspondingly faces away from the skin surface to be treated. Furthermore, the device 1 has three rollers 3, 3′, and 3″, which project beyond the housing bottom 5 by a distance H of 5 mm (see, FIG. 1a). The housing top 6 shields the rollers 3, 3′, and 3″ from above so that the device may be easily guided with the hand without unintentionally contacting the rollers 3, 3′, and 3″ with the hand. The rollers 3, 3′, and 3″ are situated in such a way that the longitudinal axis 7 of the roller 3 runs parallel to the longitudinal axis 7′, along which the two rollers 3′, 3″ are situated coaxially to one another. The longitudinal axes 7 and 7′ are at a distance of 3 cm from one another. Furthermore, the roller 3 is attached to the housing in relation to the rollers 3′ and 3″ in such a way that a projection of the roller 3 onto the axis 7′ lies between the rollers 3′ and 3″ (see, FIG. 1b) and the rollers 3, 3′, and 3″ are situated in a V-shape relative to one another (see, FIG. 1c), in such a way that the individual widths b of the line of contact of the outer peripheral surfaces 2, 2′, and 2″ of each roller 3, 3″, and 3″ combine to form a coherent total width B (see, FIG. 1c). In the present device 1, the individual width b is 2 cm and the total width B is accordingly 6 cm. In comparison to a device which has only one of the rollers 3, 3′, and 3″ described, through this special arrangement of the rollers 3, 3′, and 3″, a skin region having a defined area may be rolled over uniformly in a third of the time.

An axle 8 runs through each roller 3, 3′, or 3″ along its longitudinal axis 7, 7′. The axle projections 9 of the axle 8 project beyond both sides of the rollers 3, 3′, or 3″ in each case in the axial direction. Struts 10, which extend in a direction toward the skin surface to be treated, are attached in pairs to the housing 4 and are used to attach the rollers 3, 3′, 3″ to the housing 4. In order to be able to accommodate the axle projections 9 of axles 8, a recess 11 is formed in each strut 10 in the end facing toward the skin surface.

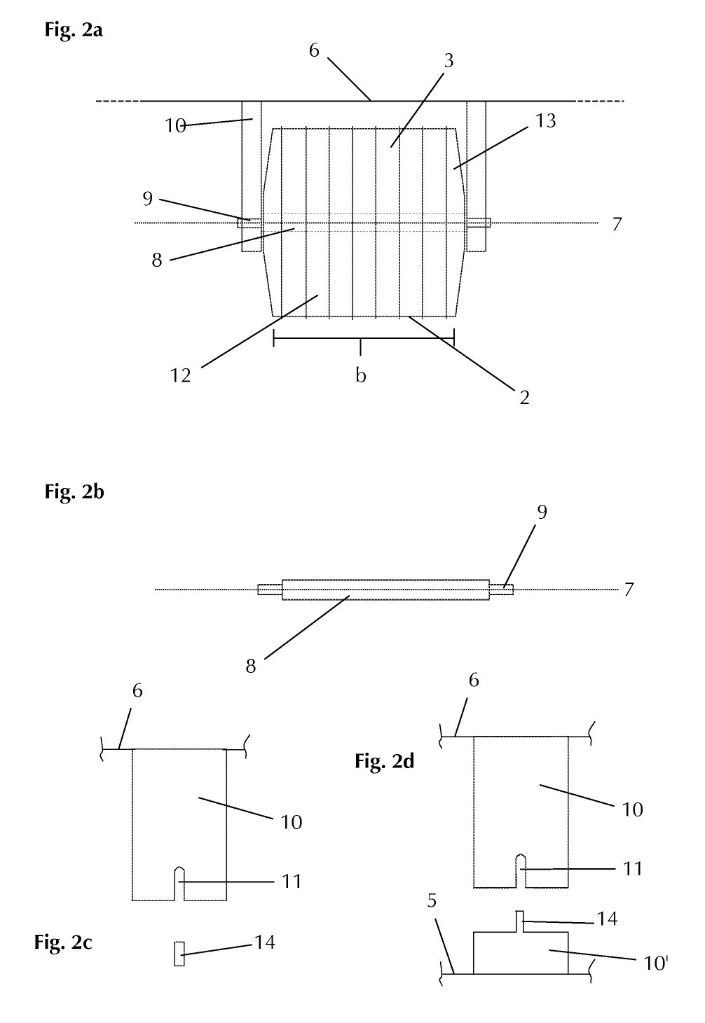

FIG. 2a is a detail enlargement in the area of two struts 10 attached to the housing 4 which carry the roller 3. For this purpose, the axle 8 runs through the roller 3 along its longitudinal axis 7. In detail, the roller is constructed from a stack of seven disks 12, which are adjoined on both sides in the axial direction by the end disks 13. The needles (not shown here) are mounted between the individual disks 12 and the end disks 13 and project beyond the outer peripheral surface 12 of the roller 3. The more detailed construction of the individual disks 12 and the end disks 13 may be seen in FIGS. 3a, 3b, and 3c.

The rollers 3, 3′, and 3″ have the axle 8, whose preferred implementation is shown in FIG. 2b, running through them along the respective longitudinal axis 7, 7′. The axle 8 has the axle projections 9 on its axial ends, which have a narrower diameter by 1 mm than the middle part of the axle 8, which has a diameter of 2 mm. This special arrangement in which the axle projections 9 have a smaller axle diameter than the remaining axle 8, prevents the axle 8 from slipping laterally out of the recess 11 of the struts 10, since the recess 11 of the strut shown in FIG. 2c has a width which essentially corresponds to the diameter of the axle projections 9. Therefore, the axle 8 may only be guided into the recess 11 in the area of the axle projection 9. The strut 10 shown has its dimensions designed so that it may absorb the forces resulting as the device 1 is guided on the skin surface and allow safe and sustainable positioning of the roller 3. For this purpose, the strut 10 is 2 mm thick in the axial direction of the roller and has a width of 10 mm perpendicular to the axial direction of the roller.

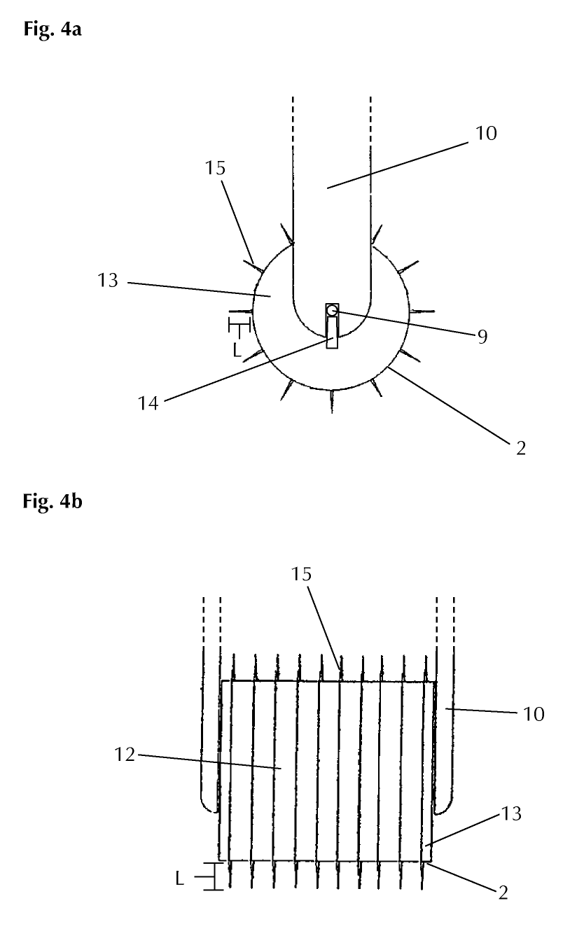

In order to prevent the axle projections 9 from sliding out of the recesses 11 in the direction toward the skin surface during transport of the device, for example, fixing means 14 are used, which are shown in FIG. 2c. The width of the fixing means 14 is selected so that they may just be inserted into the recess 11, and thus, close the recess 11 in the direction of the skin surface. After the insertion of the axle projections 9, the fixing means 14 are pushed into the recess 11 and glued there, for example (cf. FIG. 4a). Alternatively, a set screw can be used.

FIG. 2d shows an alternative embodiment of FIG. 2c. The upper strut 10 attached to the top 6 of the housing corresponds to that of FIG. 2c. The fixing means 14 is made integral with a lower strut 10′ which extends from the bottom 5 of the housing.

The stacked construction of the identically constructed rollers 3, 3′, and 3″ made of individual disks 12 can be recognized in particular in FIG. 1b. This special construction allows the uniform distribution of the needles in parallel rows both over individual rollers, such as roller 3, and also over the entirety of the rollers 3, 3′, and 3″.

FIG. 3a shows an example of a disk 12 equipped with needles 15 in a front view of one of the disk surfaces. The disk 12 is produced from plastic through injection molding. Multiple depressions 16 are provided on the surface of the disk 12, which extend radially from the disk interior up to its outer edge. The angle between neighboring depressions 16 measured from the disk center point out is 30° in each case. A needle 15 is laid into each of the depressions 16 and attached using an adhesive. A centering projection 17, similar to a toothed gear, is located in the center of the disk 12, and projects beyond the remaining surface of the disk 12. The troughs between the individual teeth of the toothed gear simultaneously form a stop 18 for the needles 15, whose rear ends lie in the troughs. The stops 18 prevent the needles 15 from being able to be pushed under pressure into the interior of the disk 12.

All needles 15 have essentially the same length. They project by the length L beyond the outer peripheral surfaces 2, 2′, and 2″ of the rollers 3, 3′, and 3″ or each individual outer peripheral surface of the disks 12. The needles 15 taper to a point on the outside. In this tip area 19, the needles have a diameter of 0.08 mm. Furthermore, the length L corresponds to the desired penetration depth of the needles into the skin to be treated. This is shown in FIG. 3a where the dashed line in the lower part of the drawing schematically represents the skin surface. As the roller is rotated, it moves forward over the skin surface and the needles 15 penetrate into the skin surface in sequence.

In order to be able to guide an axle through a roller or through the disks 12, respectively, each of the disks 12 has a through hole 20 in the disk center. The axle 8 shown in FIG. 2b is pushed through this hole and subsequently mounted in the recesses 11 of the strut pair 10.

FIG. 3b shows the disk 12 from FIG. 3a from the rear side. Instead of the centering projection 17 in the form of the toothed collar, this side of the disk 12 has a correspondingly shaped centering depression 21. If multiple disks 12, as shown in FIG. 2a, for example, are unified to form a roller 3, the centering projection 17 comes to rest in the centering depression 21 of a neighboring disk 12. It is thus not only ensured that the neighboring disks are centered exactly to one another, but also that the needles 15 of neighboring disks come to rest in a specific orientation relative to one another. Situating the needles 15 of neighboring disks in rows which run parallel to the axles 8 of individual rollers 3 is possible. However, it is also just as possible to situate needles 15 of neighboring disks staggered relative to one another.

In the individual roller shown in FIG. 2a, seven of the disks 12 shown in FIG. 3a are situated parallel to one another. The front termini of the rollers 3 are formed by the end disks 13, which differ in their implementation from the middle disks 12. For example, the surfaces of the end disks 13 pointing outward are smooth and do not have depressions or projections. The end disks 13 also differ from one another. One of the end disks has an interior surface design which corresponds to that shown in FIG. 3a. The centering projection 17 in the form of the toothed collar engages in a formfitting way in the corresponding centering depression 21 of the neighboring middle disk 12. In addition, the depressions 16 are equipped with needles 15 and form the eighth row of needles of the roller shown in FIG. 2a. Overall, this roller thus has 96 needles.

The second end disk 13 is shown in FIG. 3c which shows the surface facing toward the interior of the roller 3. In the area around the through opening 20, the end disk 13 has a depression 22. This depression 22 is just large enough to be able to accommodate the toothed collar projection 17 of the neighboring middle disk 12.

The individual disks, which form the roller 3 equipped with needles 15 of the device 1 according to the present invention, are attached to one another using an adhesive. The same adhesive which is also used for gluing the needles 15 into the depressions 16 on the disks 12 is expediently used for this purpose.

Through the special construction of the rollers formed from stacked disks 12, the needles, as shown in FIGS. 4a & 4b, may be situated in rows of needles which are continuous over all disks. Alternatively, however, a lateral offset of the needles 15 with a staggered arrangement relative to one another is also possible. For the sake of clarity, the proportions of the needles are significantly enlarged in FIGS. 4a & 4b.

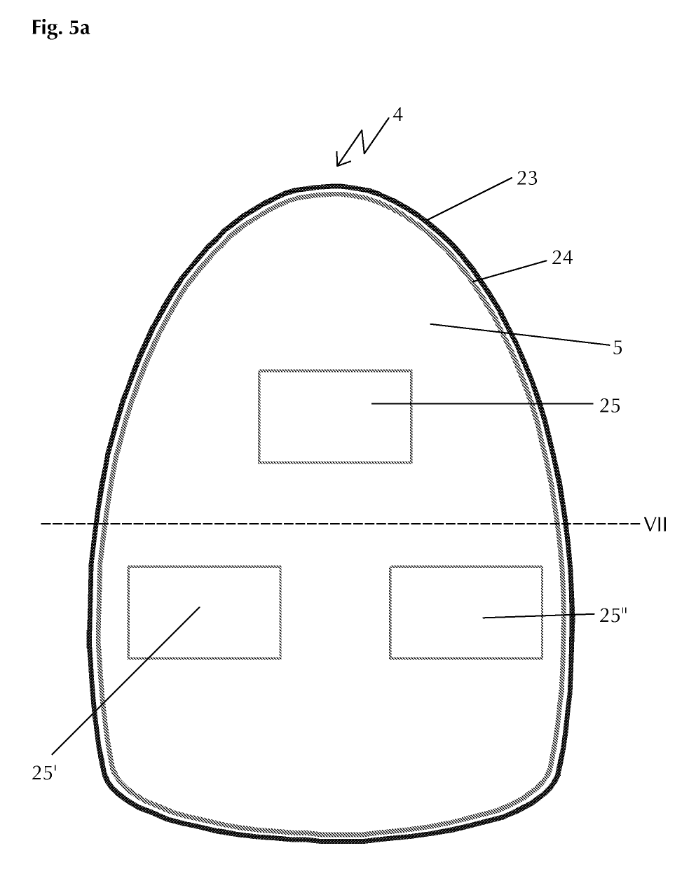

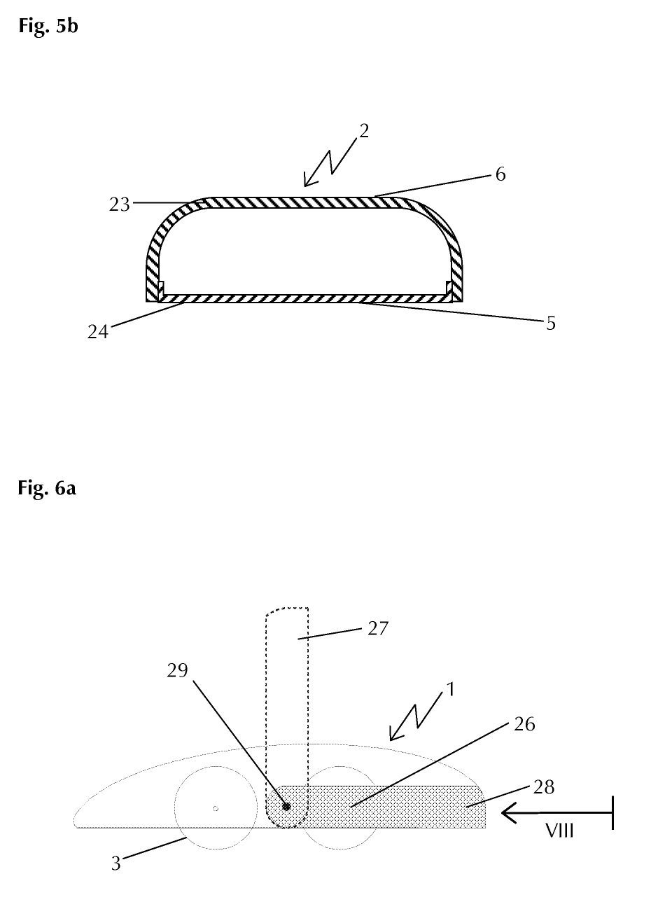

FIG. 5a shows a top view of the bottom 5 of the housing 4. As can be seen in FIG. 5b, the housing 4 comprises an upper part 23 and a lower part 24. The lower part has three rectangular openings 25, 25′, and 25″ in its surface, through which the rollers 3, 3′, and 3″ (not shown here) may penetrate. The openings 25, 25′, and 25″ are laid out in such a way that the clearance between them and the projecting rollers 3, 3′, and 3″ is as small as possible.

To connect the upper part 23 to the lower part 24, the side walls of the upper part 23 enclose the side walls of the lower part 24. A cross-section of the housing 2 along dashed line VII from FIG. 5a is shown for this purpose in FIG. 5b. Upper and lower parts may be connected to one another by catch connections, for example.

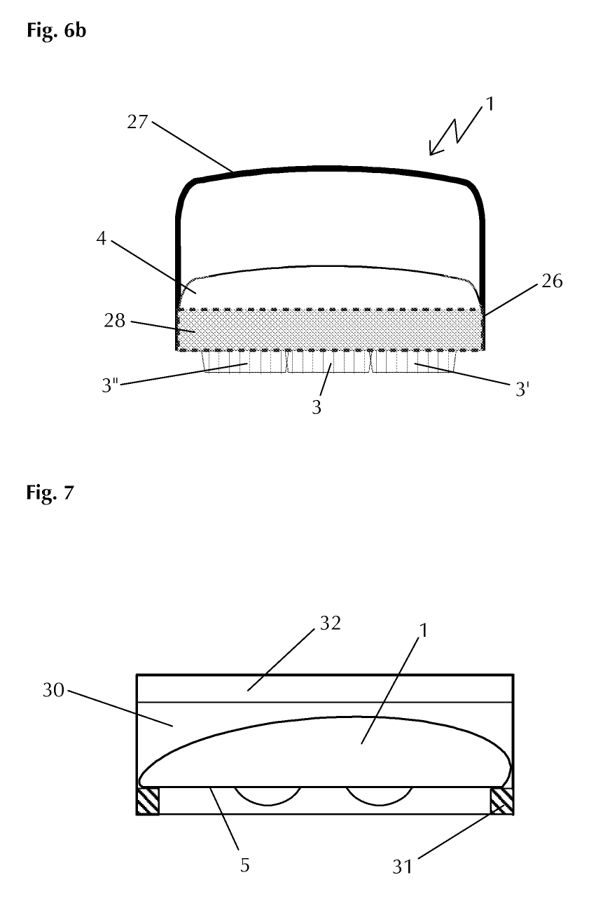

In order to make transport and safe guiding of the device 1 easier, a bow 26 is attached to the housing 4 (compare side phantom view of FIG. 6a). The bow 26 may be pivoted over the housing top 6 and moved freely back-and-forth between a swung-up operating position 27 and the pivoted-down storage position 28. The bow 26 is attached here via protrusions 29 located on each of the bow ends, which are inserted into round holes attached to the housing 4. The protrusions 29 extend at a height of 5 mm from the bow interior. In the embodiment shown, the bow 26 has sufficient elasticity so that the bow 26 may be bent apart reversibly. This special arrangement has the advantage that the bow 26 may be removed from the housing 4 and attached again as frequently as desired.

FIG. 6b shows the arrangement described in FIG. 6a in a rear view along arrow VIII from FIG. 6a.

The schematic side sectional view of FIG. 7 shows a transport container 30, into which the device 1 is inserted. Support elements 31 are attached along the edges between the side walls and the container floor of the transport container 30, on which the bottom 5 of the housing 4 comes to rest at a height which prevents the needles 15 of the rollers 3, 3′, 3″ from contacting the bottom 5 of the housing 4. Furthermore, a fitted cover 32 is provided for the transport container 30, in order to be able to close the transport container 30 on top. In addition to the simple transport function, the transport container 30 may additionally be used as a cleaning bath by accommodating standard cleaning and disinfection liquids, such as alcoholic solutions and diluted hydrogen peroxide solutions, etc. For this purpose, the container is expediently produced from a material which is chemically stable in relation to cleaning and disinfection liquids of this type. This is preferably a suitable plastic.

What I claim is:

1. A device for enabling an active ingredient to be introduced into a subject's skin, comprising:
at least two rollers mounted to rotate around a respective longitudinal axis, and
multiple needles are mounted which taper outward to a point at least in a tip area, the needles being mounted in the rollers projecting radially outward from an outer peripheral surface of the rollers,
wherein said at least two said rollers are situated parallel to one another with axially neighboring rollers being laterally offset relative to one another such that a projection of one of the rollers perpendicular to the longitudinal axis thereof comes to rest on an extension of the longitudinal axis of the neighboring parallelly situated roller without needle-bearing areas of the outer peripheral surfaces of both rollers overlapping in an axial direction.

2. The device according to claim 1, wherein said at least two rollers comprises at least three rollers.

3. The device according to claim 2, wherein the longitudinal axes of every other roller are coaxial with respect to each other.

4. The device according to claim 1, wherein all of the rollers have an identical construction.

5. The device according to claim 1, wherein the needles are distributed uniformly over the outer peripheral surfaces of all rollers in circumferential and axial directions of the rollers.

6. The device according to claim 1, wherein all of the needles project the same distance from the outer peripheral surface of the rollers.

7. The device according to claim 1, wherein said distance that all of said needles project is 0.12 to 1.5 mm.

8. The device according to claim 7, wherein the needles have a diameter of 0.05 to 0.1 mm in the tip area.

9. The device according to claim 7, wherein the needles have a diameter of 0.08 mm in the tip area.

10. The device according to claim 8, wherein the needles have a diameter outside of the tip area of between 0.15 and 0.3 mm.

11. The device according to claim 1, wherein each of the rollers is formed of parallel disks between which the needles are embedded, and wherein each of the rollers has a through hole into which an axle is inserted along the longitudinal axis thereof.

12. The device according to claim 11, wherein at least one centering projection is provided on one of the disk surfaces, the at least one centering projection being engaged in a corresponding centering depression in a facing surface of a neighboring disk, the centering projection simultaneously being a stop for at least one of the needles.

13. The device according to claim 12, wherein the at least one centering projection the shape of a toothed gear and projects from a center area of the disk.

14. The device according to claim 1, wherein the device further comprises a housing, which carries the rollers and shields them on a side facing away from a skin-contacting side of the rollers.

15. The device according to claim 14, wherein the housing has pairs of spaced apart struts which project inwardly from an inner side of the housing, the struts of a strut pair having recesses which receive axle end sections which project axially out of opposite ends of a respective one of the rollers.

16. The device according to claim 14, wherein the rollers are attached to the housing in such a way that at least the tip area of the needles projects beyond a plane spanned by a skin-facing surface of the housing.

17. The device according to claim 15, further comprising fixing means for securing said axle end sections of the roller in the recesses of the struts.

18. The device according to claim 14, wherein the housing comprises an upper part and a lower part, the lower part having openings through which at least the tips of the needles pass.

19. The device according to claim 18, wherein the struts are attached to the upper part and the fixing means are attached to the lower part.

20. The device according to claim 14, further comprising a bow which extends parallel to the longitudinal axes of the rollers and which is pivotable between a stored position against that housing an active position extending above the top of the housing.

21. The device according to claim 14, wherein the housing is made of a plastic material.
